(19) United States
(12) Patent Application Publication
(10) Pub. No.: US 2023/0098443 A1
(43) Pub. Date: Mar. 30, 2023

(54) SELF-HEATING FOLDING MASSAGE TABLE

(71) Applicant: Jiangsu Mingteng Health Products Co., Ltd, Yancheng (CN)

(72) Inventor: Chuankui Du, Jinshan (CN)

(73) Assignee: Jiangsu Mingteng Health Products Co., Ltd, Yancheng (CN)

(21) Appl. No.: 18/073,573

(22) Filed: Dec. 2, 2022

(30) Foreign Application Priority Data
Sep. 16, 2022 (CN) 202222465613.5

(51) Int. Cl. A61G 13/10; A61G 13/00; A61F 7/00

(52) U.S. Cl. CPC A61G 13/105; A61G 13/009; A61G 13/10; A61F 7/007; A61G 2203/46; A61G 2210/90; A61F 2007/0096; A61F 2007/0086

(57) ABSTRACT

The present disclosure is applicable to the technical field of massage apparatuses, and provides a self-heating folding massage table. The self-heating folding massage table includes a rigid planner surface, a sponge cushion, an electric heating mechanism, a circuit board, a transformer, a temperature sensing device, a controller, an upholstery cover, and leg supports. An operating voltage of the electric heating element is a safe operating voltage lower than 36 V, so that the safety of a user can be ensured, and the fault rate of a product is lower. The temperature sensing device is added in the electric heating mechanism to prevent fusing of the electric heating element or a fire caused by an extremely high local temperature.

### TECHNICAL FIELD

The present disclosure relates to the technical field of massage apparatuses, in particular, to a self-heating folding massage table.

### BACKGROUND

Existing massage tables are usually simple and are generally composed of a rigid planner surface, sponge and leather, but they ignore the use experience of users. Users feel cold in such tables in winter, and easily catch a cold. In the design of the existing U.S. Pat. No. 6,918,144B2, an electric heating mechanism is directly connected to an external socket through a wire, and a voltage of 110-230 V is made to the electric heating mechanism. The electric heating mechanism is possibly easily short-circuited due to moisture, or it is also easy to cause a risk of electric shock to users due to a damage of an insulation layer or the melting of the insulation layer caused by an extremely high temperature of the electric heating mechanism. The voltage of 110-230 V also causes short-circuit and thermal fusing phenomena more easily. In addition, no temperature sensing device is mentioned in the design of the existing U.S. Pat. No. 6,918,144B2. Without a temperature sensing device, a temperature sensor fails to sense a real-time temperature of the electric heating mechanism, which possibly causes the risk of overheating of the electric heating mechanism.

### SUMMARY

An embodiment of the present disclosure is to provide a self-heating folding massage table, and aims to solve the following problems: A user feels cold in an existing massage table in winter and easily catches a cold. By adding an electric heating function, a more comfortable warm feeling will be brought to the user.

The present disclosure is achieved by a self-heating folding massage table. The self-heating folding massage table includes:

a rigid planner surface, a sponge cushion, an electric heating mechanism, a circuit board, a transformer, a temperature sensing device, a controller, an upholstery cover, and leg supports, wherein the rigid planner surface is of a sectioned structure; hinges for folding the rigid planner surface are mounted between sectioned rigid planner surfaces; the leg supports are mounted below the rigid planner surface; the leg supports get close to be folded around roots of the leg supports towards the rigid planner surface; the sponge cushion includes at least two layers, and the sponge cushion is arranged on the rigid planner surface; an upper layer of the sponge cushion is in contact with the body of a client; the sponge cushion is folded together with the sectioned rigid planner surface; the electric heating mechanism is arranged between the two layers of the sponge cushion; an electric heating element capable of generating heat after being powered up is arranged in the electric heating mechanism; the electric heating element is connected to the circuit board through a wire; the circuit board is connected with the transformer through a wire; the transformer supplies power to the circuit board; and the circuit board supplies power to the electric heating element in the electric heating mechanism.

In a further technical solution, an operating voltage of the electric heating element in the electric heating mechanism is a safe voltage lower than 36 V.

In a further technical solution, a plurality of temperature sensing devices are mounted in the electric heating mechanism; the temperature sensing devices may sense a temperature of the electric heating element; and the temperature sensing devices are connected to the circuit board through wires and communicate with the circuit board, so that the circuit board senses a real-time heating temperature of the electric heating element.

In a further technical solution, the transformer may convert an externally supplied voltage of 110-230 V into a safe voltage lower than 36 V, and supplies power to the circuit board.

In a further technical solution, corresponding to the sectioned design of the rigid planner surface, the electric heating mechanism is also divided into a plurality of sections, and the plurality of sections of the electric heating mechanism are connected through wires.

In a further technical solution, the controller is connected to the circuit board through a wire; the controller is provided with a button; the button is in communication connection with the circuit board through a wire; and the circuit board may be controlled through the button.

In a further technical solution, the circuit board pre-stores a working program; the circuit board receives an information instruction of the button; and the circuit board starts to supply power to the electric heating element according to the pre-stored working program, and controls the magnitude of the current or stops supplying the power.

In a further technical solution, the upholstery cover is upholstered on the sponge cushion and the electric heating mechanism, and fixes the sponge cushion and the electric heating mechanism on the rigid planner surface; and an insulation protection layer is arranged between the upholstery cover and the sponge cushion.

Compared with the prior art, the present disclosure has the beneficial effects:

According to the self-heating folding massage table provided by the present disclosure, an electric heating function can be turned on by controlling the button before massage. Compared with the existing U.S. Pat. No. 6,918,144B2, the self-heating folding massage table is additionally provided with the circuit board to control the electric heating function. Furthermore, the electric heating mechanism with a low voltage lower than 36 V is used, which is safer to users, and the users have a softer heating feel. Short circuit is avoided, and the fault rate is decreased. By the adding of the temperature sensing devices, the circuit board can sense the temperature of the electric heating mechanism in real time, so that the circuit board better controls the electric heating mechanism and prevents the electric heating mechanism from being overheated.

### BRIEF DESCRIPTION OF THE DRAWINGS

FIG. 1 is a front view of a self-heating folding massage table provided according to an embodiment of the present disclosure;

FIG. 2 is a side view of a self-heating folding massage table provided according to an embodiment of the present disclosure;

FIG. 3 is a top view of a self-heating folding massage table provided according to an embodiment of the present disclosure;

FIG. 4 is a bottom view of a self-heating folding massage table provided according to an embodiment of the present disclosure; and

FIG. 5 is an electric control schematic diagram of a self-heating folding massage table provided according to an embodiment of the present disclosure.

In the drawings: 1: rigid planner surface; 2: bottom-layer sponge; 3: upper-layer sponge; 4: electric heating mechanism; 5: temperature sensing device; 6: electric control box; 7: transformer; 8: controller; 9: upholstery cover; 10: electric heating element; 12: circuit board; and 13: button.

### DETAILED DESCRIPTION OF THE EMBODIMENTS

In order to make the objectives, technical solutions and advantages of the present disclosure clearer, the present disclosure is further described in detail below with reference to accompanying drawings and embodiments. It should be understood that the specific embodiments described here are merely to illustrate and explain the present disclosure, and not intended to limit the present disclosure.

Specific implementation of the present disclosure is described in detail below in combination with specific embodiments.

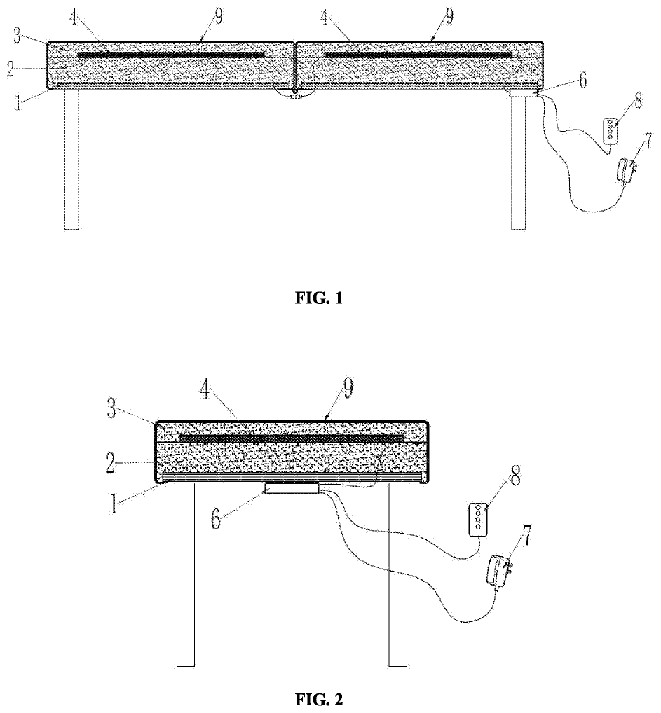

As shown in FIG. 1 to FIG. 3, a self-heating folding massage table provided according to one embodiment of the present disclosure includes:

a rigid planner surface 1, a sponge cushion, an electric heating mechanism 4, an electric control box 6, a transformer 7, a controller 8, and an upholstery cover 9. The sponge cushion includes bottom-layer sponge 2 and upper-layer sponge 3. The rigid planner surface 1 is a rigid plywood divided into a left rigid planner surface 1 and a right rigid planner surface 1. Two hinges are mounted between the left and right rigid planner surfaces 1. The left and right parts of the rigid planner surface 1 may be folded around the hinges. Leg supports are mounted below the rigid planner surface 1. The leg supports may rotate around joint parts between the leg supports and the rigid planner surface 1 and are folded towards the bottom of the rigid planner surface 1. The bottom-layer sponge 2 adopts an elastic sponge material and is laid on the rigid planner surface, and the upper-layer sponge 3 adopts a soft sponge material and is laid on the bottom-layer sponge 2. The electric heating mechanism 4 is laid in an interlayer between the upper-layer sponge 3 and the bottom-layer sponge 2. An electric heating element 10 is arranged in the electric heating mechanism 4. The electric heating mechanism 4 is formed by sewing two layers of cloth. The electric heating element 10 is mounted in an interlayer between the two layers of cloth of the electric heating mechanism 4. The electric heating element 10 is located in a center region of the electric heating mechanism 4 and is uniformly distributed.

The electric heating element 10 may generate heat after being powered up. An operating voltage of the electric heating element 10 is lower than 36 V. Using a safe voltage lower than 36 V can avoid the risk of electric shock to users.

As shown in FIG. 1 to FIG. 5, an electric control box 6 is mounted below the rigid planner surface 1. A circuit board 12 is mounted inside the electric control box 6.

As shown in FIG. 1 to FIG. 4, the transformer 7 is connected to an external power supply. The transformer 7 may convert a high voltage of 110-230 V into a safe voltage lower than 36 V. The transformer 7 is connected to the circuit board 12 in the electric control box 6 through a wire. The transformer 7 supplies electric energy to the circuit board 12 in the electric control box 6.

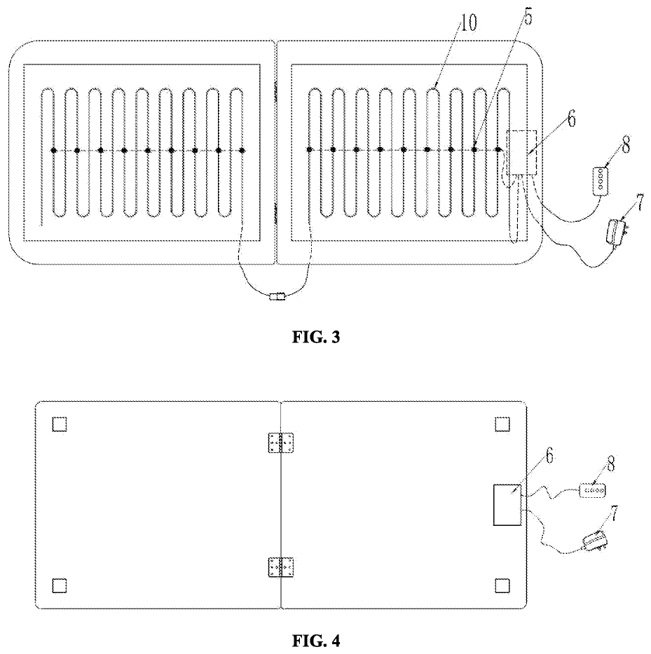

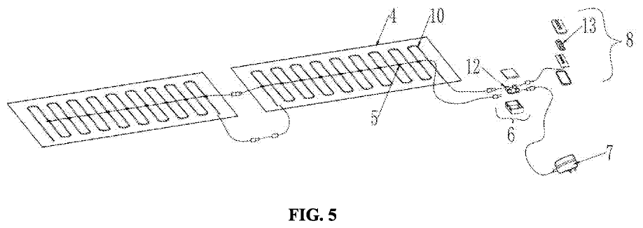

As shown in FIG. 3 and FIG. 5, the electric heating mechanism 4 is divided into two pieces. The electric heating elements 10 in the two electric heating mechanisms 4 are connected through a wire. The built-in electric heating element 10 in one of the two electric heating mechanisms 4 is connected to the circuit board 12 through a wire. The transformer 7 supplies electric energy to the circuit board 12. The circuit board 12 supplies power to the built-in electric heating element 10 in the electric heating mechanism 4, thus forming an electric loop.

As shown in FIG. 3 and FIG. 5, a plurality of temperature sensing devices 5 are mounted in the electric heating mechanism 4. The temperature sensing devices 5 may be implemented using a pulse width modulation (PWM) manner or a negative temperature coefficient (NTC) temperature sensor. The temperature sensing devices 5 are connected to the circuit board 12 through wires and communicate with the circuit board 12. The temperature sensing devices 5 may monitor a temperature of the electric heating element 10 in the electric heating mechanism 4 and transmit monitored temperature information to the circuit board 12.

The circuit board 12 pre-stores a working program. The circuit board 12 supplies electric energy to the electric heating element 10 in the electric heating mechanism 4 according to the pre-stored working program. The electric heating element 10 generates heat after being powered up. The temperature sensing devices 5 monitor the temperature of the electric heating element 10 in the electric heating mechanism 4. When the temperature of the electric heating element 10 does not reach a target temperature, the circuit board 12 continues to supply power to the electric heating element 10. When the temperature of the electric heating element 10 reaches or exceeds the target temperature, the circuit board 12 stops supplying power to the electric heating element 10.

The heating capacity of the heating element 10 varies with the current provided by the circuit board 12, the stronger the current, the greater the heat.

The upper-layer sponge 3 has a moderate thickness, which can play a role of dispersing and conducting the heat. The heat can uniformly and softly reach a user lying in supine or prone position on the massage table.

As shown in FIG. 1 to FIG. 5, the controller 8 is provided with a button 13. The controller 8 is connected to the circuit board 12 through a wire. The button 13 communicates with the circuit board 12 through a wire.

The user sends an instruction to the circuit board 12 by means of operating the button 13 of the controller 8. The user can set a target heating temperature through the button of the controller 8. The circuit board 12 supplies power to the built-in electric heating element 10 in the electric heating mechanism 4 according to the target heating temperature and the pre-stored program, and controls the magnitude of a current, thus controlling the electric heating element 10 to generate heat.

As shown in FIG. 1 to FIG. 2, the upholstery cover 9 is upholstered on the bottom-layer sponge 2, the electric heating mechanism 4 and the upper-layer sponge 3, and fixes them on the rigid planner surface 1. An insulation protection layer is arranged between the upholstery cover 9 and the upper-layer sponge 3. The insulation protection layer adopts a plastic film, which can achieve an insulation protection effect and also achieve waterproof and moisture proof effects.

In the embodiments of the present disclosure, the user can set the target temperature by means of operating the button 13 of the controller 8, and turn on and turn off a heating function.

The above descriptions are only the preferred embodiments of the present disclosure, and are not intended to limit the present disclosure. Any modifications, equivalent replacements and improvements that are made within the spirit and principle of the present disclosure shall fall within the protection scope of the present disclosure.

What is claimed is:

1. A self-heating folding massage table, comprising:
a rigid planner surface, a sponge cushion, an electric heating mechanism, a circuit board, a transformer, a temperature sensing device, a controller, an upholstery cover, and leg supports,
wherein the rigid planner surface is of a sectioned structure; hinges for folding the rigid planner surface are mounted between sectioned rigid planner surfaces; the leg supports are mounted below the rigid planner surface; the leg supports get close to be folded around roots of the leg supports towards the rigid planner surface; the sponge cushion includes at least two layers, and the sponge cushion is arranged on the rigid planner surface; an upper layer of the sponge cushion is in contact with the body of a user; the sponge cushion is folded together with the sectioned rigid planner surface; the electric heating mechanism is arranged between the two layers of the sponge cushion; an electric heating element capable of generating heat after being powered up is arranged in the electric heating mechanism; the electric heating element is connected to the circuit board through a wire; the circuit board is connected with the transformer through a wire; the transformer supplies power to the circuit board; and the circuit board supplies power to the electric heating element in the electric heating mechanism.

2. The self-heating folding massage table according to claim 1, wherein an operating voltage of the electric heating element in the electric heating mechanism is a safe voltage lower than 36 V.

3. The self-heating folding massage table according to claim 1, wherein a plurality of temperature sensing devices are mounted in the electric heating mechanism; the temperature sensing devices sense a temperature of the electric heating element; the temperature sensing devices are connected to the circuit board through wires and communicate with the circuit board; and through communication between temperature sensing devices with the circuit board, the circuit board to sense a real-time heating temperature of the electric heating element.

4. The self-heating folding massage table according to claim 1, wherein the transformer converts an externally supplied voltage of 110-230 V into a safe voltage lower than 36 V, and supplies power to the circuit board.

5. The self-heating folding massage table according to claim 1, wherein the electric heating mechanism comprises a plurality of sections; the plurality of sections of the electric heating mechanism are used cooperatively with the sectioned rigid planner surface; and the plurality of sections of the electric heating mechanism are connected through wires.

6. The self-heating folding massage table according to claim 1, wherein the controller is connected to the circuit board through a wire; the controller is provided with a button for controlling the circuit board; and the button is in communication connection with the circuit board through a wire.

7. The self-heating folding massage table according to claim 1, wherein the circuit board pre-stores a working program used for controlling a power supply state and a magnitude of a supply current.

8. The self-heating folding massage table according to claim 1, wherein the upholstery cover is upholstered on the sponge cushion and the electric heating mechanism, and fixes the sponge cushion and the electric heating mechanism on the rigid planner surface, and an insulation protection layer is arranged between the upholstery cover and the sponge cushion.
